(19) United States
(12) Patent Application Publication
(10) Pub. No.: US 2018/0117263 A1
(43) Pub. Date: May 3, 2018

(54) INJECTABLE MATERIAL INJECTION DEVICE COMPRISING A CANNULA THAT SLIDES INSIDE A NEEDLE, AND SYSTEM USING SUCH A DEVICE

(71) Applicant: Peter CUMBO, Vence (FR)

(72) Inventor: PETER CUMBO, VENCE (FR)

(21) Appl. No.: 15/575,765

(22) PCT Filed: May 19, 2016
(86) PCT No.: PCT/FR2016/051184

§ 371 Date: Nov. 20, 2017

(30) Foreign Application Priority Data
May 19, 2015 (FR) 1554466

(51) Int. Cl. A61M 5/32; A61M 25/06; A61M 5/34

(52) U.S. Cl. CPC A61M 5/3287; A61M 5/3286; A61M 5/329; A61M 5/3293; A61B 17/3415; A61M 5/347; A61M 2005/3201; A61M 2210/04; A61M 25/065

(57) ABSTRACT

An injectable material injection device includes a cannula that slides inside a hypodermic needle. The hypodermic needle includes a beveled tip. The cannula includes a base that is configured to establish a sealed connection.

### TECHNICAL FIELD OF THE INVENTION

The present invention is aimed at an injection device for injecting an injectable product and at a system for injecting an injectable product.

The present invention can be used to perform any injection, for medical or esthetic purposes, notably for subcutaneous injections. The invention finds a particularly advantageous application in the injection of injectable fillers for esthetic purposes.

### PRIOR ART

It is known practice in the prior art to perform injections of fillers, such as hyaluronic acid or calcium hydroxyapatite or even collagen, on the face using a simple syringe comprising a hollow needle able to pierce the skin. The filler contained in the syringe is injected under the skin at various points to obtain a desired esthetic effect. This conventional method has the disadvantage that ten to fifteen needle pricks have to be made, in order to cover the entire face, thereby causing the patient considerable pain and discomfort. The risks of edema and of hematoma are also increased, because of the high number of needle pricks.

It is known practice from the prior art to use a cannula in order to reduce the number of needle pricks required. The skin is first of all pierced using a hollow needle of suitable diameter, then the cutaneous orifice thus produced is identified by the appearance of a drop of blood. A cannula is then inserted into the cutaneous orifice then the cannula is guided toward the various sites at which the filler is injected. This method has the disadvantage of often making the introduction of the cannula difficult, as the orifice created using the needle is very often difficult for the practitioner to identify, notably if no drop of blood appears and the cannula may be difficult to insert, meaning that the practitioner is obliged to make several attempts at this, causing greater tissue trauma and an increased risk of bleeding, pain and edema, all of which are sources of discomfort to the patient.

None of the current systems is able simultaneously to meet all the requirements, namely of allowing products to be injected at multiple sites within the tissues from a single point at which the skin is pierced using a simple action: one single device and one single action.

### OBJECT OF THE INVENTION

The present invention seeks to overcome all or some of these disadvantages.

To this end, a first aspect of the present invention relates to an injection device for injecting an injectable product, which comprises a cannula sliding in a hypodermic needle comprising a beveled tip, the cannula comprising a base configured to establish a sealed connection.

What is meant by an injectable product is any liquid, particularly liquids that can be injected into the human body. The product may be hyaluronic acid, calcium hydroxyapatite, collagen for example.

Thanks to these measures, an operator can, in a single action, pierce the skin of a patient using a hollow needle, then deploy within the tissues a cannula with a soft, atraumatic end, so that an injectable product can be applied therein. These measures eliminate the step that consists in attempting to insert the cannula through the orifice made beforehand in the skin using an independent hypodermic needle, the cannula then being deployed through the lumen of the guide needle. In addition, the operator may perform the operations of piercing the skin with the needle and of inserting the cannula with the device kept in contact with the skin. Such measures differ from the solutions of the prior art in which the operations of piercing the skin and of inserting the cannula require a piercing to be made using a first device, then require the first device to be removed and hands moved to take hold of a second device and perform the step of inserting the cannula.

In some embodiments, the tip of the hypodermic needle comprises a main bevel and a rear bevel.

By virtue of these measures, the piercing of the skin by the main bevel of the needle tip is easy, while at the same time being atraumatic when the cannula is deployed, this being because of the rear bevel that effaces the tip which becomes flush with the wall of the cannula. Thus, during manipulations of the cannula, the tip of the needle does not cause the tissue any trauma.

In some embodiments, the hypodermic needle comprises a first screw thread and the cannula comprises a second screw thread configured to be screwed to the first screw thread.

By virtue of these measures, the needle is fixed to the cannula. The operator can easily attach and detach the cannula with respect to the needle by screwing or unscrewing. Also, thanks to these measures, the operator is able, by screwing the cannula into the needle, to control the length of cannula extending beyond the tip of the needle. Precise control over the length of cannula extending beyond the tip of the needle is important, particularly during the step of inserting the cannula into and moving it around in the tissues.

In some embodiments, the device comprises a blocking means configured to block the cannula with respect to the hypodermic needle in a predetermined position.

By virtue of these measures, the blocking device makes it possible to prevent the needle from sliding with respect to the cannula. The blocking means allows the cannula to be blocked with respect to the needle in at least one predetermined position.

In some embodiments, the device comprises at least one visual reference indicating the length of that part of the cannula that is extending beyond the tip of the needle.

By virtue of these measures, the operator can know the length of cannula extending beyond the tip of the needle simply by looking at the visual reference. The visual reference is situated for example on the screw thread of the cannula or on the insertion end of the cannula.

In some embodiments, the base comprises a syringe connector of Luer Lock type.

By virtue of these measures, the base of the cannula can be connected in a sealed manner notably to a syringe. These measures allow a quick, reliable and sealed connection between the device that forms the subject of the invention and any other medical component that has a connector of Luer Lock type. A medical component comprising a connector of Luer Lock type may be a transfusion tube or alternatively a perfusion tube.

In some embodiments, the cannula has an insertion end of rounded shape.

By virtue of these measures, the end that is introduced into the patient's skin has a round non-beveled and non-cutting end which can penetrate the tissues without causing lesions to vessels and nervules in particular, reducing the discomfort to the patient during the injection session and reducing the risk of bruising.

A second aspect of the present invention relates to a system for injecting a product which comprises a product injection device that forms the subject of the present invention.

Since the objects, advantages and special features of the product injection system bearing the device that forms the subject of the present invention are similar to those of the device that forms the subject of the present invention, they are not repeated here.

### BRIEF DESCRIPTION OF THE FIGURES

Further advantages, objects and special features of the present invention will become apparent from the non-limiting description which follows of at least one particular embodiment of the devices, methods and systems that form the subjects of the present invention, with reference to the attached drawings, in which:

FIG. 1 depicts, schematically and in side view, one particular embodiment of the device that forms the subject of the present invention;

FIG. 2 depicts, schematically and in side view, one particular embodiment of the device that forms the subject of the present invention;

FIG. 3 depicts, schematically and in cross section, one particular embodiment of the tip of the needle of the device that forms the subject of the present invention;

FIG. 4 depicts, schematically and in perspective, one particular embodiment of the tip of the needle of the device that forms the subject of the present invention; and

FIG. 5 depicts, schematically and in side view, one particular embodiment of a system bearing the device that forms the subject of the present invention.

### DETAILED DESCRIPTION OF SOME EMBODIMENTS OF THE INVENTION

The present description is given by way of nonlimiting example, each feature of one embodiment being able to be combined with any other feature of any other embodiment advantageously.

Note that the term “a” is used throughout in the sense of “at least one”.

Note at this point that the figures are not drawn to scale.

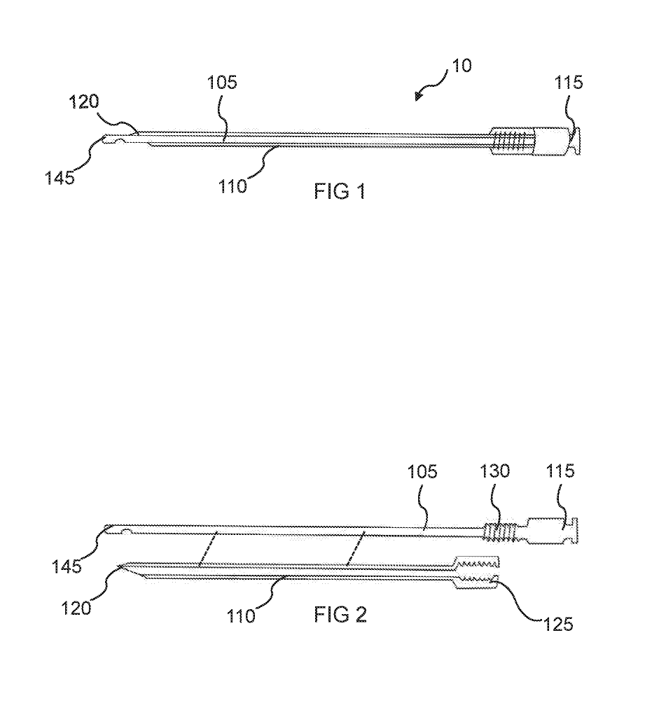

FIG. 1 shows an injectable-product injection device 10 which comprises a cannula 105 sliding in a hypodermic needle 110 comprising a bevel tip 120, the cannula 105 comprising a base 115 configured to establish a sealed connection. The cannula comprises a tube made of plastic or of metal, straight or curved, allowing air or an injectable product to pass through an orifice, either natural or made by the practitioner. For preference, the cannula 105 is made of metal, of straight elongate shape and configured to inject an injectable product. In some embodiments, the cannula 105 is formed of steel, stainless steel or titanium.

In some embodiments, the cannula 105 comprises an insertion end 145 configured to be inserted through a hole made in the skin and, at the other end, a base comprising a screw thread 130. The insertion end 145 and the screw thread 130 are described in FIG. 2. The cannula tube has a predetermined diameter configured to be suited to the product that is to be injected. In some embodiments, the cannula 105 has a diameter of 1.24 mm (an abbreviation for millimeter), 0.81 mm, 0.71 mm, 0.63 mm, 0.56 mm, 0.46 mm or 0.30 mm. In preferred embodiments, the cannula 105 has a diameter comprised between 0.56 mm and 0.30 mm.

The cannula 105 comprises a base 115 configured to make a sealed connection with a syringe, a piece of transfusion or perfusion equipment for example. In some embodiments, the base 115 comprises a syringe connection of Luer type. It will be recalled here that Luer connections are standardized sealed connectors notably described in European standard NF EN 20594-1 published in 1993 and French standard ISO 594-1 published in 1986. In other embodiments, the base 115 comprises a reverse Luer type of connector.

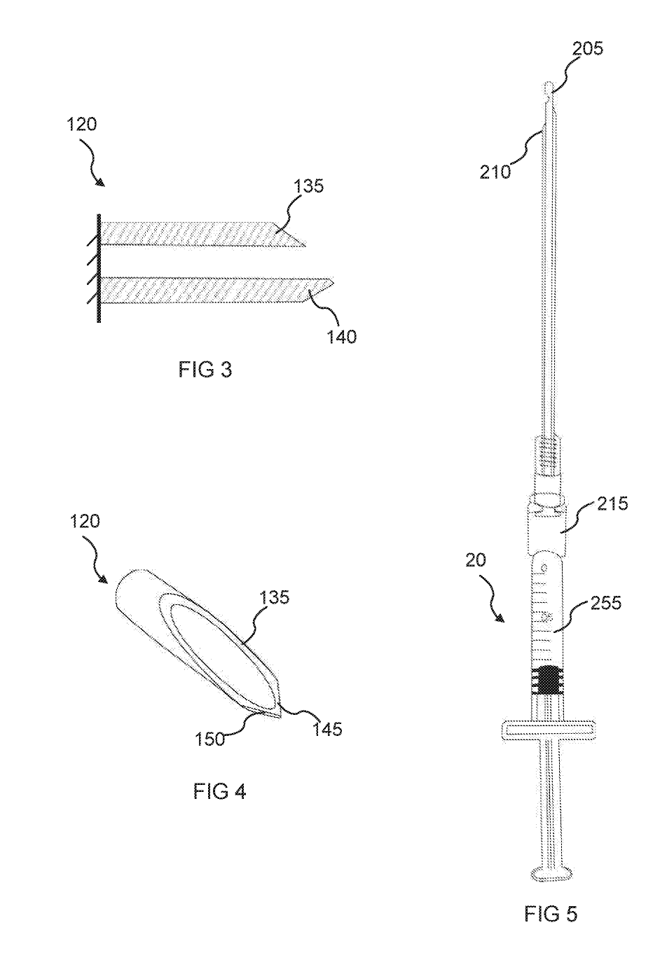

The hypodermic needle 110 comprises a tip 120 depicted in FIGS. 3 and 4, configured to pass through the skin by piercing it. The hypodermic needle 110 comprises a tube made of plastic or of metal. For preference, the hypodermic needle 110 is a metal tube of a diameter greater than that of the cannula. In some embodiments, the hypodermic needle 110 comprises a screw thread 125 described in FIG. 2.

The two hollow tubes of the cannula 105 and of the hypodermic needle 110 are arranged in concentric circles, the cannula 105 being contained inside the hypodermic needle 110. The cannula 105 is able to slide translationally with respect to the hypodermic needle 110 in the sense of the slideway connection formed by the assembly formed of the cannula 105 and the hypodermic needle 110.

For preference, the cannula 105 is longer than the hypodermic needle 110. In some embodiments, the length of the cannula 105 is comprised between 25 mm and 75 mm. For preference, the length of the hypodermic needle 110 is 5 mm shorter than the length of the cannula 105.

In some embodiments, the hypodermic needle 110 is very short. In this embodiment, the length of the hypodermic needle 110 is comprised between five and ten millimeters, and allows a significant length of cannula 105 to extend beyond the tip 120 of the hypodermic needle 110. In some embodiments, the length of cannula 105 that can extend beyond the tip 120 of the hypodermic needle is greater than 25 mm.

In some embodiments, the device 10 comprises at least one visual reference indicating the length of that part of the cannula that is extending beyond the tip 120 of the hypodermic needle 110.

FIG. 2 shows an exploded view of the product injection device 10 which comprises a sliding cannula 105 and a hypodermic needle 110.

The cannula 105 comprises an insertion end 145 configured to be slipped under the skin and to inject an injectable product. In some embodiments, the insertion end 145 comprises an opening configured to allow the product injected by the cannula to exit. In some embodiments, the opening of the insertion end 145 is set back from the tip of the insertion end 145. In some embodiments, the distance between the tip of the insertion end 145 and the opening is greater than one millimeter.

In some preferred embodiments, the cannula 105 comprises an insertion end 145 of rounded shape.

For preference, the insertion end 145 of the cannula 105 is configured to be atraumatic. The insertion end 145 of the cannula 105 is a rounded shape that has no cutting edge.

In some embodiments, the hypodermic needle 110 comprises a first screw thread 125 and the cannula comprises a second screw thread 130 configured to be screwed to the first screw thread 125. The first screw thread 125 and the second screw thread 130 are configured so that the cannula 105 can be screwed from a piercing position in which the cannula 105 does not protrude beyond the tip of the hypodermic needle 110 into an injection position in which the insertion end 145 of the cannula 105 extends beyond the tip of the hypodermic needle 110.

In preferred embodiments, the cannula 105 comprises a screw thread 130 on its external surface and the needle 110 comprises a screw thread 125 on its internal surface. In other words, the screw thread 125 on the internal surface of the hypodermic needle 110 is a tapped thread.

In some embodiments, the needle 110 comprises a screw thread on its external surface and the cannula 105 comprises a screw thread on its internal surface.

In some embodiments, the first screw thread 125 and the second screw thread are triangular metric threads. In other embodiments, the first screw thread 125 and the second screw thread 130 are helical threads.

As a preference, the first screw thread 125 and the second screw thread 130 have a length of between 3 mm and 15 mm.

In some embodiments, the device 10 comprises a blocking means configured to block the cannula 105 with respect to the hypodermic needle 110 in at least one predetermined position. In some embodiments, the blocking means is a shoulder on the hypodermic needle 110. In preferred embodiments, the predetermined positions at which the cannula 105 is blocked in the hypodermic needle 110 by the blocking means include at least one first blocking position in which the cannula does not extend beyond the tip of the hypodermic needle 110 and at least one blocking position in which the length of the part of the cannula 105 that extends beyond the tip of the hypodermic needle 110 is greater than 2 mm.

The blocking means is configured to prevent translational movement between the cannula 105 and the hypodermic needle 110. In some embodiments, the blocking means is an articulated clamp held under tension by a spring. Thus, the operator may press the ends of the clamp to allow translational movement of the cannula 105 with respect to the hypodermic needle 110 or release to prevent translational movement of the cannula 105 with respect to the hypodermic needle 110.

FIG. 3 shows a view in section of one embodiment of the bevel tip 120 of the needle 110 of the device 10. For preference, the tip 120 of the hypodermic needle comprises a main bevel 135 and a rear bevel 140. In some embodiments, a grinding tool comprising abrasive means is used to machine the main bevel, the main bevel 135 being cut on the surface of the hypodermic needle 110 at a predetermined angle with respect to the axis of the hypodermic needle 110.

For preference, the main bevel 135 has a larger surface area than the rear bevel. For preference, the main bevel 135 has a flat surface. In some embodiments, the angle defined by the main bevel 135 with respect to the axis of the hypodermic needle 110 is less than forty degrees. For preference, the rear bevel 140 is cut on a part of the hypodermic needle 110 that is diametrically opposite that of the main bevel 135.

FIG. 4 shows a perspective view of one embodiment of the bevel tip 120 of the needle 110 of the device 10 that forms the subject of the present invention. In some embodiments, the tip of the hypodermic needle 110 comprises a main bevel 135 and two secondary bevels 145 and 150 cut one on each side of the slender part formed by the main bevel. In other words, the secondary bevels 145 and 150 are facets positioned one on each side of the main bevel.

FIG. 5 shows one embodiment of the product injection system 20 which comprises the product injection device that forms the subject of the present invention. For preference, the product injection system 20 comprises a cannula 205 sliding in a hypodermic needle 210 comprising a bevel tip, the cannula 205 comprising a base 215 configured to establish a sealed connection and a syringe 255.

In some embodiments (not depicted), the injectable-product injection system 20 comprises a cannula 205 sliding in a hypodermic needle 210 comprising a bevel tip, the cannula 205 comprising a base 215 configured to establish a sealed connection and a flexible perfusion or transfusion tube.

In some embodiments (not depicted), the system 20 comprises a cannula thrusting means allowing the cannula 205 to be made to slide translationally with respect to the hypodermic needle 210 in the direction of the slideway connection formed by the assembly of the cannula 205 and the hypodermic needle 210.

In some embodiments (not depicted), the system 20 comprises a visual indication indicative of the orientation of the plane defined by the main bevel. For preference, the visual indication is present on the base 215.

1-8. (canceled)

9. An injection device to inject an injectable product, comprising a cannula sliding in a hypodermic needle, the hypodermic needle comprising a bevel tip and the cannula comprising a base configured to establish a sealed connection.

10. The injection device as claimed in claim 9, wherein the bevel tip of the hypodermic needle comprises a main bevel and a rear bevel.

11. The injection device as claimed in claim 9, wherein the hypodermic needle comprises a first screw thread and wherein the cannula comprises a second screw thread configured to be screwed to the first screw thread.

12. The injection device as claimed in claim 9, further comprising a blocking mechanism configured to block the cannula with respect to the hypodermic needle in a predetermined position.

13. The injection device as claimed in claim 9, further comprising at least one visual reference indicating a length of a part of the cannula extending beyond the bevel tip of the hypodermic needle.

14. The injection device as claimed in claim 9, wherein the base comprises a syringe connector of Luer Lock type.

15. The injection device as claimed in claim 9, wherein the cannula comprises an insertion end of a rounded shape.

16. A system to inject an injectable product comprising an injection device as claimed in claim 9 and a syringe; and wherein the base establishing the sealed connection with the syringe.
